(19) United States
(12) Patent Application Publication
(10) Pub. No.: US 2007/0172953 A1
(43) Pub. Date: Jul. 26, 2007

(54) Mobile telecommunication terminal having electrical compass module and playing mobile game method using electrical compass module thereof

(75) Inventor: Kihak Shim, Seongnam (KR)

Correspondence Address: HARNESS, DICKEY & PIERCE, P.L.C., P.O. BOX 828, BLOOMFIELD HILLS, MI, 48303, US

(73) Assignees: SK TELECOM LTD, Seoul (KR); NEXMORE SYSTEMS INC., Seoul (KR)

(21) Appl. No.: 10/571,966

(22) PCT Filed: Sep. 15, 2004
(86) PCT No.: PCT/KR04/02351

§ 371 Date: Nov. 20, 2006

(30) Foreign Application Priority Data
Sep. 15, 2003 (KR) 10-2003-0063772

(51) Int. Cl. G01N 33/18

(52) U.S. Cl. 436/41; 436/39; 436/9

(57) ABSTRACT

The present invention relates to mobile communication terminal including an electronic compass module and a method for playing a network mobile game by using the electronic compass module. The method for playing a mobile game by using a mobile communication terminal with an electronic compass module embedded therein, comprising the steps of: (a)providing a mobile game list embedded in the mobile communication terminal and determining if a selected mobile game is a stand-alone mobile game or a network mobile game; (b) executing the selected mobile game in the mobile communication terminal or gaining access to a wireless Internet game server via a wireless Internet so that the wireless Internet game server executes the selected mobile game; (c) controlling a movement of a user-controlled character in the mobile game under execution based on control data which is generated depending on a movement of the mobile communication terminal; and (d) transmitting and displaying a game screen, on which the user-controlled character is moved, to the mobile communication terminal on a real-time basis to execute the mobile game. The present mobile communication terminal is used to control the user-controlled character precisely and easily.

### TECHNICAL FIELD

The present invention relates to a method for playing a game by using a mobile communication terminal incorporating an electronic compass module therein; and, more specifically, to a mobile communication terminal including an electronic compass module which is operated by the same principle as that of an electronic compass and a method for playing a mobile game on the mobile communication terminal by way of converting a value outputted from the electronic compass module which varies with a movement direction of the mobile communication terminal into a three-dimensional coordinate value for use in playing the mobile game.

### BACKGROUND ART

Recently, with the rapid development in electronics, communication engineering and various technologies related to communication terminals, mobile communication terminals are endowed with diversified functions. That is to say, a user can enjoy various services including wireless Internet access, video communication, moving picture message transmission, etc., as well as voice communication by using a mobile communication terminal. Such mobile communication terminal represented by a cellular phone may overcome a greatest drawback of PC (personal computer), i.e., immobility, so that it may guarantee mobility of the user extensively.

Meanwhile, as a CPU (control processing unit) of the mobile communication terminal makes faster data processing speed possible and a color LCD (liquid crystal display) screen and a 64-chord melody level sound source are supported, it becomes possible to play various games by using the mobile communication terminal. Since a game (hereinafter, referred to as a “mobile game”) capable of being played on the mobile communication terminal may be played anytime, anywhere, and another new game may be played without replacing the mobile communication terminal unlike other game machines, the number of mobile game users has been rapidly increasing.

Specifically, although stand-alone mobile games downloaded into the mobile communication terminal has been commonly prevailed due to a poor performance of the mobile communication terminal, network mobile games that can be played with other people through the online are now being widely propagated.

FIG. 1 shows an exemplary screen of a mobile game played by using a conventional mobile communication terminal.

In FIG. 1, a game screen of the mobile game which is being played on the mobile communication terminal is displayed leftward while functions assigned to key buttons to support the mobile game are displayed rightward. Specifically, it may be known that a key button for moving a user-controlled character in the mobile game is assigned to each of an upward, a downward, a leftward, a rightward and a jumping motion. Accordingly, the user must learn the functions assigned to the key buttons which are displayed on the right portion of FIG. 1 before starting the mobile game.

For example, the key buttons assigned to the upward, the downward, the leftward, the rightward and the jumping motion must be hit continually with both thumbs of the user in order to move the user-controlled character. Further, since it is difficult to make a special motion such as “bubble attack” shown in FIG. 1 while moving the user-controlled character, a great amount of time and efforts are required to master the mobile game. Specifically, since key buttons assigned to various motions of user-controlled characters or special functions depend on respective mobile games, the difficulty in mastering the mobile games is further augmented.

Moreover, while a vertical and/or a horizontal spacing between the key buttons become narrow due to a small area of a key matrix installed on an outside of the mobile communication terminal, each thumb used to press the key buttons has a relatively wide contact area. Accordingly, other wrong key buttons adjacent to a desired key button may be frequently pressed in playing the game, thereby impeding the smooth progress of the game.

### DISCLOSURE OF THE INVENTION

It is, therefore, an object of the present invention to provide a mobile communication terminal including an electronic compass module which is operated by the same principle as that of an electronic compass and a method for playing a mobile game on the mobile communication terminal by way of converting a value outputted from the electronic compass module which varies with a movement direction of the mobile communication terminal into a three-dimensional coordinate value for use in playing the mobile game.

In accordance with a first aspect of the present invention, there is provided a mobile communication terminal for supporting a mobile game by using an electronic compass module, the mobile game being a game electronically performed by or at a mobile communication terminal, wherein the mobile game stored in the mobile communication terminal or in a wireless Internet game server is executed to play the game on a real-time basis, and an output generated from the electronic compass module embedded in the mobile communication terminal is used as direction control data for changing a movement of a user-controlled character in the mobile game under execution upward, downward, leftward and rightward from a fixed point.

In accordance with a second aspect of the present invention, there is provided a method for playing a mobile game on a mobile communication terminal supporting the mobile game by using an electronic compass module embedded therein, comprising the steps of: (a) providing a mobile game list embedded in the mobile communication terminal and determining if a selected mobile game is a stand-alone mobile game or a. network mobile game; (b) based on the result determined in the step (a), allowing the mobile communication terminal to exclusively execute the selected mobile game in the mobile communication terminal or allowing the mobile communication terminal to gain access to a wireless Internet game server via a wireless Internet so that the wireless Internet game server executes the selected mobile game; (c) controlling a movement of a user-controlled character in the mobile game under execution based on control data which is generated depending on a movement of the mobile communication terminal; and (d) transmitting and displaying a game screen, on which the user-controlled character is moved, to the mobile communication terminal on a real-time basis to execute the mobile game.

### BRIEF DESCRIPTION OF THE DRAWINGS

The above and other objects and features of the present invention will become apparent from the following description of a preferred embodiment given in conjunction with the accompanying drawings, in which:

FIG. 1 illustrates an exemplary screen of a mobile game played by using a conventional mobile communication terminal;

FIG. 2 is a block diagram for schematically showing an internal configuration of a mobile communication terminal in accordance with a preferred embodiment of the present invention;

FIG. 3 presents a block diagram for schematically showing an internal configuration of an electronic compass module in accordance with the preferred embodiment of the present invention;

FIG. 4A and 4B set forth graphs for describing a relationship between an external magnetic field in an X-axis magnetic sensor and an output value thereof and a relationship between an external magnetic filed in a Y-axis magnetic sensor and an output value thereof, respectively, in accordance with the embodiment of the present invention;

FIG. 5A and 5B illustrate a moving state of a user-controlled character when the mobile communication terminal is moved leftward and rightward in an electronic compass mode in accordance with the embodiment of the present invention;

FIG. 6A and 6B show a moving state of the user-controlled character when the mobile communication terminal is moved upward and downward in the electronic compass mode in accordance with the embodiment of the present invention; and

FIG. 7 depicts a flowchart for describing a playing sequence of a mobile game by using an electronic compass function in accordance with the preferred embodiment of the present invention.

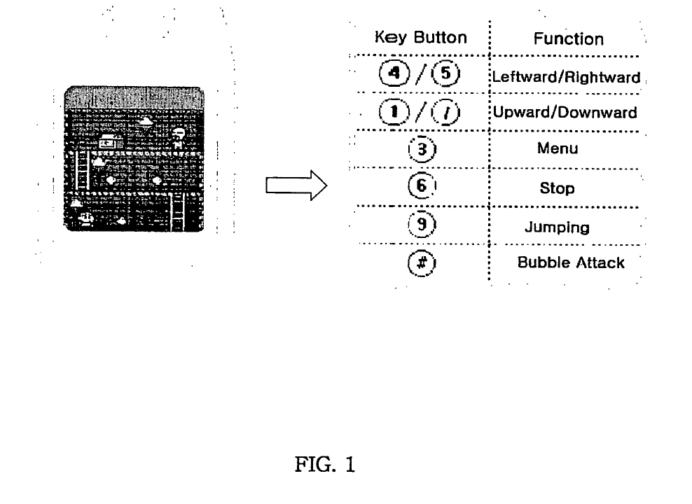

### BEST MODE FOR CARRYING OUT THE INVENTION Hereinafter, preferred embodiments of the present invention will be described in detail with reference to the accompanying drawings.

Here, like reference numerals represent like parts in various drawings. Further, it is notable that detailed description of known parts or functions will be omitted if there is a concern that the description of such parts or functions would render the technical essence of the present invention obscure.

FIG. 2 is a block diagram for schematically showing an internal configuration of a mobile communication terminal 200 in accordance with a preferred embodiment of the present invention.

The mobile communication terminal 200 includes a program memory unit 210, a parameter storage unit 211, a key input unit 212, an LCD unit 213, an electronic compass module 214, a mobile game storage unit 215, a mode state storage unit 216, a subscriber identity module 217, a microprocessor 220, a digital signal processor 230, a baseband converter 240, an RF signal processor 250, a speaker 260, a microphone 270 and an antenna 280.

Stored in the program memory unit 210 are a protocol software for processing a message transceived through a network and a compiler for processing a mobile game in accordance with the preferred embodiment of the present invention. The compiler serves to compile and execute mobile game contents coded in a programming language such as C⁺⁺, embedded visual C⁺⁺ and JAVA. Since every programming language used to code mobile game contents comes with its own compiler, in case of executing specific mobile game contents, file information of the mobile game contents is analyzed to obtain information on the programming language in which the mobile game contents are coded so that an appropriate compiler may be used to execute the mobile game contents.

Moreover, incorporated in the program memory unit 210 is a predetermined wireless Internet browser which allows the mobile communication terminal 200 to gain access to a specific server such as a wireless Internet server via a wireless Internet to execute a network mobile game or download mobile game contents. The wireless Internet browser installed in the mobile communication terminal 200 may be a WAP (wireless application protocol) browser coded in WML (wireless markup language), a Mobile Explorer coded in m-HTML (Microsoft-HTML), a Compact Netfront coded in c-HTML (Compact-HTML), or the like.

Stored or capable of being stored in the parameter storage unit 211 are various parameters to be used in a synchronous, an asynchronous and a fourth-generation communication system defined by 3GPP (the 3^{rd} generation partnership project), 3GPP2, ITU (international telecommunication union), OHG (operator harmonization group) and so on to cause the mobile communication terminal to execute audio call and/or data communication. Accordingly, the protocol software stored in the program memory unit 210 uses the various parameters stored in the parameter storage unit 211 to modulate and demodulate audio signals and/or data signals transceived by the mobile communication terminal 200.

The key input unit 212 is provided with a number of key buttons for use in inputting numbers such as telephone numbers or characters. Typically, such key buttons include twelve number keys (0 to 9, *, #), a plurality of function keys, a multiplicity of cursor displacement keys, a scroll key and so forth. Thus, the user may manipulate the number keys, the function keys, direction keys and so on provided on the key input unit 212 so as to select and play a desired mobile game.

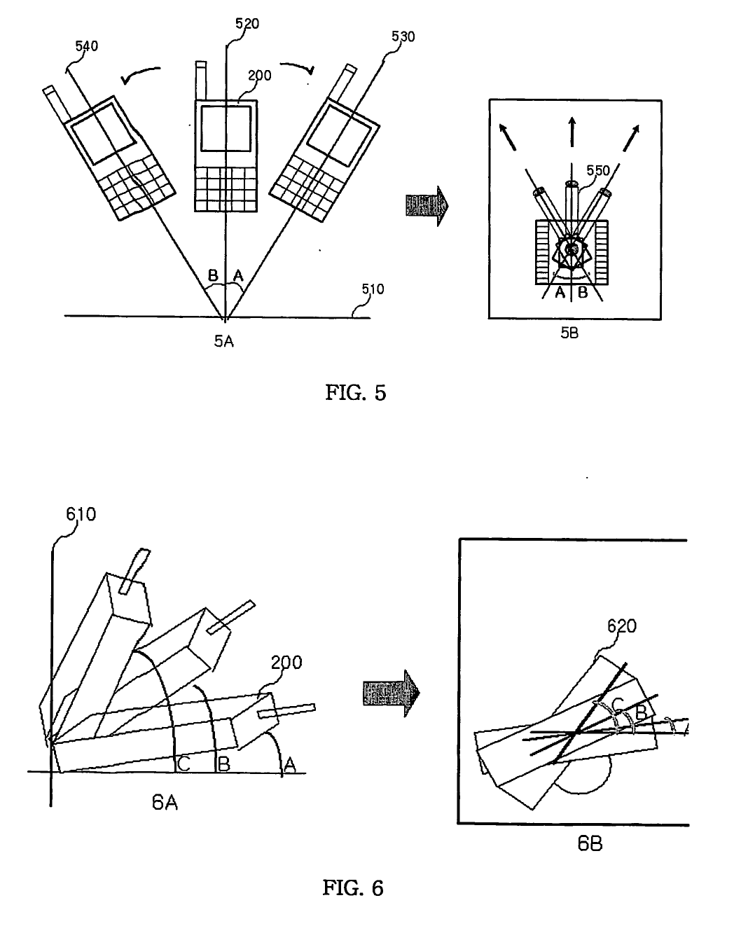

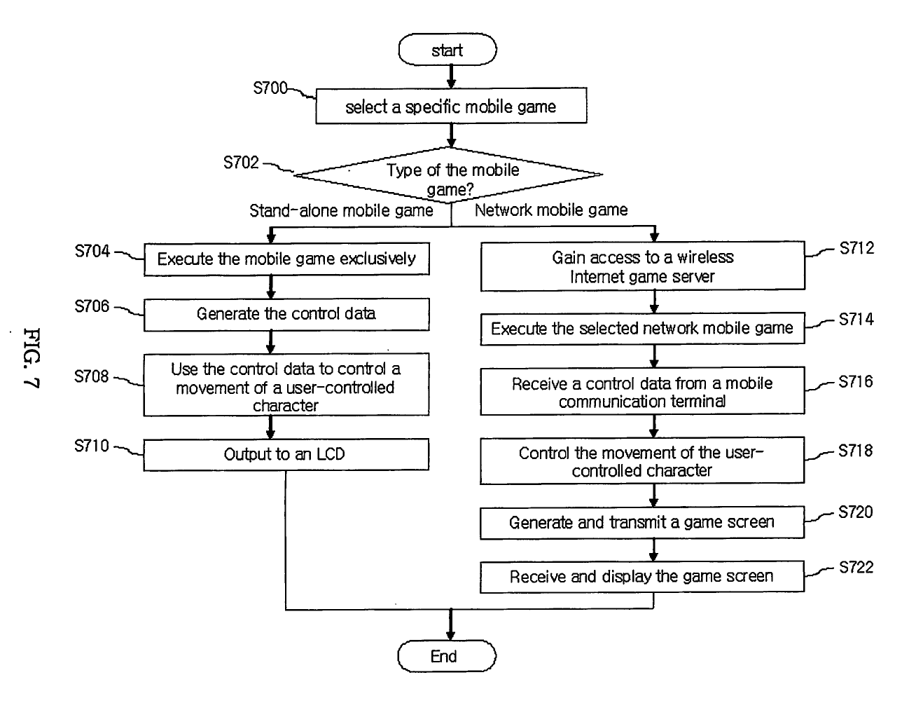

Meanwhile, in accordance with the embodiment of the present invention, the key buttons provided on the key input unit 212 can be irrelevant to the movement of an image (hereinafter, referred to as a “user-controlled character”) that can be moved by an user under the mobile game. That is to say, in accordance with the embodiment of the present invention, even though the user can manipulate the specific key buttons provided on the key input unit 212 to move the user-controlled character, the user can move the mobile communication terminal itself to move the user-controlled character. In other words, the user-controlled character may be moved in proportion to a moving direction and a moving angle by which the mobile communication terminal 200 is moved. Hereinafter, the movement of the mobile communication terminal 200 will be described in further detail with reference to FIGS. 5 and 6 in accordance with the embodiment of the present invention.

The LCD unit 213 shows operational states of the mobile communication terminal 200 including a residual battery capacity of a battery, a receiving intensity of a radio wave, date and time. Furthermore, in accordance with the embodiment of the present invention, the LCD unit 213 serves to display various mobile-game-related screens including a game screen, movements of the user-controlled character, a game score and a game ranking when the mobile game is being played on the mobile communication terminal 200.

The electronic compass module 214 incorporates therein a magnetic sensor or a geomagnetic sensor so that the electronic compass module 214 may detect a variation in a geomagnetic field depending on a moving distance and/or a moving direction, by which the mobile communication terminal 200 is moved, to generate an output signal related with the variation in the geomagnetic field. It is preferable that the electronic compass module 214 is disposed horizontally with respect to the mobile communication terminal 200 to guarantee a precise operation thereof. Accordingly, the installation place of the electronic compass module 214 may be varied depending on whether the mobile communication terminal 200 is of a flip type or a folder type. For example, with regard to a flip-type mobile communication terminal, since the key input unit 212 and the LCD unit 200 are installed on a same plane so that the electronic compass module 214 can be equilibrated with respect to the mobile communication terminal 200 wherever it is installed, the installation place of the electronic compass module 214 may not be important. Since, however, in case of a folder-type mobile communication terminal, the key input unit 212 and the LCD unit 213 may be angled with each other by a predetermined angle under the execution of the mobile game, it is preferable that the electronic compass module 214 is mounted at a body portion of the mobile communication terminal 200, in which the key input unit 212 communicated with both hands of the user is provided.

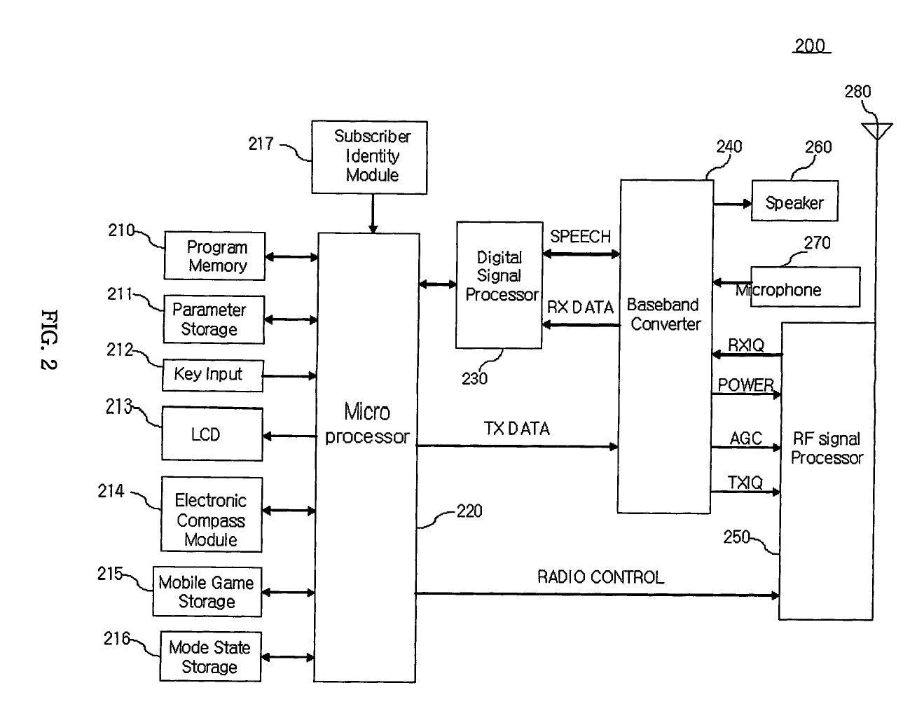

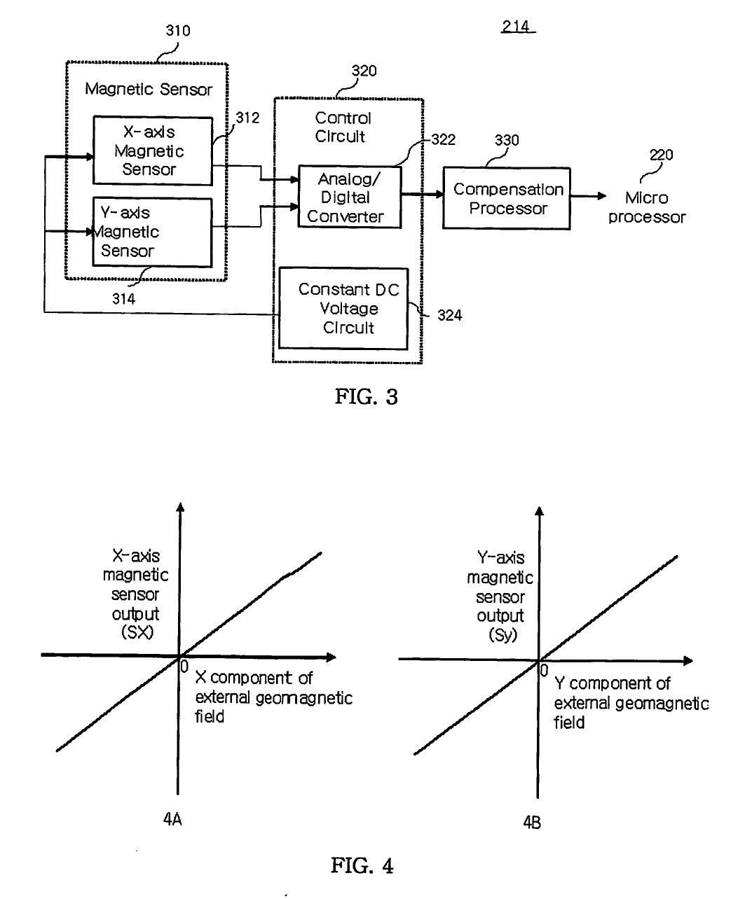

The internal configuration of the electronic compass module 214 in accordance with the preferred embodiment of the present invention will be described later in further detail with reference to FIG. 3.

The mobile game storage unit 215 stores therein at least one mobile game that is downloaded from a certain wireless Internet game server through an access to the wireless Internet or received with other methods by the mobile communication terminal 200. Meanwhile, in order to apply the technical spirit of the present invention to a mobile game, there is a user-controlled character in the mobile game, and a movement of the user-controlled character is also required, so that a shooting game and so on may be preferable. Here, the movement of the user-controlled character refers to a movement by which an angle formed between a reference axis such as a horizontal axis or a vertical axis and the user-controlled character at a fixed position is varied about the reference axis upward, downward, leftward or rightward.

Referring to FIGS. 5 and 6, there are shown such movements of the user-controlled character in accordance with the embodiment of the present invention.

Meanwhile, the mobile game employing the technical sprit of the present invention is a game for supporting a mode (hereinafter, referred to as an “electronic compass mode”) in which a value of an output signal outputted from the electronic compass module 214 is used to control the movement of the user-controlled character. Specifically, it is more preferable that the mobile game in accordance with the embodiment of the present invention supports both the electronic compass mode and a mode (hereinafter, referred to as a “key matrix mode”) in which the key buttons are used to control the movement of the user-controlled character, and the mobile game also supports a function for allowing a user to select one of the two modes in case of executing a certain mobile game.

The mode state storage unit 216 stores a current operation mode of the mobile communication terminal 200 selected by the key input unit 212 as a state flag (for example, 0, 1, 2, . . .). That is, the microprocessor 220 assigns an identified state flag to each mode in order to distinguish a standby mode, a call mode, a stand-alone mobile game mode and a network mobile game mode, and updates the mode state storage unit 216.

The subscriber identify module (SIM) 217 stores therein a mobile identification number (MIN), an electrical serial number (ESN), a personal security key and various data required to operate the mobile communication terminal. The SIM 217, which is also called as an SIM card because it has a card shape adapted to be inserted into a slot inside the mobile communication terminal, serves as an interface between the mobile communication terminal and a wired or wireless communication network. Various integrated circuit (IC) cards can be used instead of the SIM card, and an inner chip capable of being embedded in the mobile communication terminal may be used to realize the function of the subscriber identity module 217 without a separate card.

The microprocessor 220 controls the overall operation of the mobile communication terminal 200 by referring to the state flag stored in the mode state storage unit 216. When a key value related to a request for execution of a stand-alone mobile game is inputted from the key input unit 212, the microprocessor 220 displays a list of stand-alone mobile games stored in the mobile game storage unit 215 on the LCD unit 213. Meanwhile, when a key value related to a request for execution of a network mobile game is inputted from the key input unit 212, the microprocessor 220 gains access to a predetermined wireless Internet game server via the wireless Internet, receives a game list of network mobile games therefrom and displays the game list on the LCD unit 213.

If a network mobile game is selected by the user, the microprocessor 220 plays the game while communicating game data with the wireless Internet game server via the wireless Internet on a real-time basis. That is to say, the microprocessor 220 transmits not only game-control key values inputted from the key input unit 212 but also game-control data received from the electronic compass module 214 to the wireless Internet game server via the wireless Internet on the real-time basis. The wireless Internet game server, which receives the game-control key values and/or the game-control data from the mobile communication terminal 200 via the wireless Internet, controls the movement of the user-controlled character and the like based on rules of the game being played on the mobile communication terminal 200. Moreover, the wireless Internet game server provides game screen data of the game being played to the mobile communication terminal 200 via the wireless Internet on the real-time basis, thereby allowing the user to check the game screen data.

Meanwhile, the technology for supporting a network mobile game by using the mobile communication terminal, the wireless Internet, the wireless Internet game server and so forth is well known to those skilled in the art, and, therefore, the detailed description thereof will be omitted.

Further, the microprocessor 220 may clarify what kind of modes are supported by the stand-alone mobile game or the network mobile game and display it on a game list screen. For example, <key> may be displayed for a game that supports a key matrix mode only; <compass>, for a game that supports an electronic compass mode only; and <key & compass>, for a game that supports both the key matrix mode and the electronic compass mode.

When the user selects a mobile game that supports both the key matrix mode and the electronic compass mode from the game list screen, the selected mobile game provides a mode selection screen to allow the user to select a certain mode prior to starting the game. If a mobile game that supports only an electronic compass mode is selected or if an electronic compass mode is selected from the mode selection screen, the microprocessor 220 operates the electronic compass module 214 and controls the movement of a user-controlled character in the mobile game under being executed by using values of output signals transmitted from the electronic compass module 214. Furthermore, the microprocessor 220 transmits digital data (TX DATA) for use in performing various functions requested through the key input unit 212 to the baseband converter 240.

The digital signal processor (hereinafter, referred to as a “DSP”) 230 is a digital signal processing processor for encoding and/or decoding a speech signal, serving as an equalizer to eliminate multiple channel noises and performing audio data processing function. Further, the DSP 230 exchanges speech data (SPEECH) with the baseband converter 240 and receives digital data (RX DATA) from the baseband converter 240.

The baseband converter 240 converts signals communicated between the DSP 230 and the RF signal processor 250, the speaker 260 and the microphone 270 into baseband signals and serves as a digital to analog converter (DAC) and an analog to digital converser (ADC). Moreover, the baseband converter 240 delivers transmission data (TXIQ) to the RF signal processor 250 and controls a power (POWER) of the RF signal processor 250 or automatically controls an automatic gain control (AGC) of the RF signal processor 250. Then, the baseband converter 240 receives a received signal (RXIQ) from the RF signal processor 250.

The RF signal processor 250 demodulates and amplifies the RF signal received from the RF antenna 280, and modulates the transmission signal provided from the baseband converter 240 to transmit the modulated signal into a wave propagation space. The speaker 260 receives audio data outputted from the currently played mobile game through the baseband converter 240 and outputs the audio data as audible sounds, and the microphone 270 converts a speech input of the user into an electric signal.

Meanwhile, the mobile communication terminal 200 in accordance with the embodiment of the present invention may be a PDA (personal digital assistant), a cellular phone, a PCS (personal communication service) phone, a hand-held PC, a GSM (global system for mobile) phone, a W-CDMA (wideband CDMA) phone, a CDMA-2000 phone or a MBS (mobile broadband system) phone. Here, the MBS phone refers to a phone to be used in a fourth generation system currently being discussed.

FIG. 3 is a block diagram for schematically showing the internal configuration of the electronic compass module 214 in accordance with the preferred embodiment of the present invention.

The electronic compass module 214 in accordance with the preferred embodiment of the present invention includes a magnetic sensor 310, a control circuit 320, a compensation processor 330 and so forth.

The magnetic sensor 310 is an element for outputting a specific signal depending on a direction and a magnitude of an external magnetic field, and includes an X-axis magnetic sensor 312 and a Y-axis magnetic sensor. 314. In general, the magnetic sensor is a ring-shaped magnetic permalloy with high magnetic permeability around which an exciting coil is wounded along the entire circumference thereof and the X-axis magnetic sensor 312 and the Y-axis magnetic sensor 314 serving as detection coils are wounded along diametrical directions to be perpendicular to each other, respectively.

Meanwhile, a geomagnetic field, i.e., the Earth's magnetic field, is a magnetic field facing north from south. If a main body of the mobile communication terminal 200 has a front side which is substantially flat and faces upward, the X-axis magnetic sensor 312 and the Y-axis magnetic sensor 314 output sensor output signals with cosine and sine waves as the external magnetic field fluctuates.

Here, since the principle in which the magnetic sensor outputs the sensor output signal in response to the variation in the external magnetic field is well known to those skilled in the art, the detailed description thereof will be omitted.

The control circuit 320 includes an analog to digital converter (ADC) 322, a constant DC voltage circuit 324 and so forth. The control circuit 320 serves to process the output signals provided from the X-axis magnetic sensor 312 and the Y-axis magnetic sensor 314, to thereby output digital signals.

The ADC 322 receives the sensor output signal outputted from each of the X-axis magnetic sensor 312 and the Y-axis magnetic sensor 314 to convert the received signal into a digital signal. Here, the value of the digital signal outputted from the ADC 322 is determined as a vertical or a horizontal rotation angle value about the reference axis such as the horizontal or the vertical axis due to a movement of the user-controlled character during the game. Furthermore, the ADC 322 sends the converted digital signal to the compensation processor 330.

The constant DC voltage circuit 324 supplies a constant DC voltage, whose magnitude is not changed, to the X-axis magnetic sensor 312 and the Y-axis magnetic sensor 314 connected thereto, to thereby assist precise sensing operation of the magnetic sensor.

The compensation processor 330 receives a digital signal from the ADC 322 of the control circuit 320; if a compensation of the digital signal is required, the compensation processor 330 performs the compensation thereof; and transfers the compensated digital signal to the microprocessor 220. Here, when the digital signal has a negative value or is equal to or greater than 360°, it is determined that the compensation of the digital signal is required. If the compensation processor 330 determines the received digital signal to be negative, 360° may be added to the received digital signal to compensate the rotation angle value, while, if the compensation processor 330 determines the received digital signal to be equal to or greater than 360°, 360° may be subtracted from the received digital signal, thereby compensating the rotation angle value. Thus, the compensated digital signal outputted from the electronic compass module 214 is always controlled to have a value ranging from 0° to 360°.

Further, in case a threshold value for the angle of horizontal rotation or vertical rotation is predetermined in the currently played mobile game, the compensation processor 330 compares the value of the received digital signal with the threshold value, and may perform a compensation thereof in the same manner as described above. For example, if the threshold value for the angle of horizontal rotation or vertical rotation is set as 90° or 180°, digital signals which exceed 90° or 180° may be subjected to compensation for subtracting 90° or 180° therefrom.

FIG. 4A and 4B set forth graphs for describing a relationship between an external magnetic field in an X-axis magnetic sensor 312 and an output value thereof and a relationship between an external magnetic filed in a Y-axis magnetic sensor 314 and an output value thereof, respectively, in accordance with the embodiment of the present invention.

As can be known from FIG. 4, the X-axis magnetic sensor 312 outputs a sensor output value S_{X} proportional to an X component of the external magnetic field at a state where the X-axis magnetic sensor 312 is mounted in the mobile communication terminal 200. Likewise, the Y-axis magnetic sensor 314 outputs a sensor output value S_{y} proportional to a Y component of the external magnetic field at a state where the Y-axis magnetic sensor 314 is mounted in the mobile communication terminal 200. The X-axis magnetic sensor 312 and the Y-axis magnetic sensor 314 have same configuration for allowing them to output signal values proportionate to the magnitude of magnetic fields in respective predetermined directions, and are disposed to be perpendicular to a detecting direction of the magnetic field on a single chip of the magnetic sensor 310.

FIG. 5A and 5B describes a moving state of a user-controlled character 550 when the mobile communication terminal 200 is moved leftward and rightward in an electronic compass mode in accordance with the embodiment of the present invention.

Referring to FIG. 5A, it is assumed that the mobile communication terminal 200 is moved rightward by an angle A about a line perpendicular to the reference axis, i.e., the horizontal axis 510. Here, each of reference numerals 520, 530 and 540 represents a rotation axis of the mobile communication terminal 200. Accordingly, when the mobile communication terminal 200 is moved rightward by the angle A, its precise meaning is that the angle formed between two rotation axes 520 and 530 is the angle A.

If the mobile communication terminal 200 is moved rightward by the angle A, the Y-axis magnetic sensor 314 embedded in the mobile communication terminal 200 generates a sensor output signal in proportion to a magnitude change of the external field and provides the sensor output signal to the microprocessor 220 via the ADC 322 and the compensation processor 330. The microprocessor 220 reads a compensated digital signal provided from the compensation processor 330 and moves the user-controlled character 550 in the game under execution in the same direction and by the same angle as those by which the mobile communication terminal 200 is moved, as shown in FIG. 5B. Here, the moving angle of the user-controlled character 550 may be set to be identical to the real moving angle of the mobile communication terminal 200, but it may be also magnified or scaled down at a predetermined ratio by multiplying the real moving angle of the communication terminal 200 by a preset weight. Here, it is apparent that the mobile communication terminal 200 moved leftward by an angle B can be explained by the same principle as the rightward movement of the mobile communication terminal by the angle A has been explained.

Accordingly, by moving the mobile communication terminal 200 leftward or rightward with reference to the horizontal axis, the user can easily execute a leftward and a rightward angular adjustment of the use-controllable character in the real mobile game.

FIG. 6A and 6B shows a moving state of the user-controlled character 620 when the mobile communication terminal 200 is moved upward and downward in the electronic compass mode in accordance with the embodiment of the present invention.

Referring to FIG. 6A, it is assumed that the mobile communication terminal 200 is moved upward by angles A, B and C about a line perpendicular to the reference axis, i.e., the vertical axis 610.

If the mobile communication terminal 200 is moved upward by the angle A, the X-axis magnetic sensor 312 embedded in the mobile communication terminal 200 generates a sensor output signal in proportion to magnitude variations of the external field and provides the sensor output signal to the microprocessor 220 via the ADC 322 and the compensation processor 330. The microprocessor 220 reads a compensated digital signal provided from the compensation processor 330 and moves the user-controlled character 620 in the game under execution in the same direction and by the same angle as those by which the mobile communication terminal 200 is moved, as shown in FIG. 6B. Here, as described above in FIG. 5, the moving angle of the user-controlled character 620 may be set to be identical to the real moving angle of the mobile communication terminal 200, but it may be also magnified or scaled down at a predetermined ratio by multiplying the real moving angle of the communication terminal 200 by a preset weight.

Accordingly, by moving the mobile communication terminal 200 upward or downward with reference to the vertical axis, the user can easily execute a upward and a downward angular adjustment of the use-controllable character in the real mobile game.

Referring to FIG. 7, there is shown a flowchart for describing a playing sequence of a mobile game by using an electronic compass function in accordance with the preferred embodiment of the present invention.

A playing process of the mobile game in accordance with a preferred embodiment of the present invention relates to a method in which the mobile game has been stored in the mobile communication terminal 200 and, based on a type of the mobile game selected by the user, the mobile communication terminal 200 allows the mobile game to be executed executively or allows the mobile game to be executed communicating with the wireless Internet game server. In other words, if a stand-alone mobile game is selected among mobile games stored in the mobile communication terminal 200, the selected mobile game is executed exclusively on the mobile communication terminal 200 and, if a network mobile game is selected, the selected mobile game is executed communicating with the wireless Internet game server.

A user selects a mobile game function mounted on the mobile communication terminal 200 to be provided with a game list screen and allows a selection key value for a desired specific game to be inputted (S700). Here, the game list includes the stand-alone mobile game and the network mobile game. The mobile communication terminal 200 uses the selection key values to be inputted to determine if the selected mobile game is the stand-alone mobile game or the network mobile game (S702).

If the selected mobile game is determined to be the stand-alone mobile game in the step S702, the microprocessor 220 embedded in the mobile communication terminal 200 executes the selected stand-alone mobile game exclusively (S704). The mobile communication terminal 200 generates control data which varies with a movement of the mobile communication terminal 200 itself (S706). Specifically, if the mobile communication terminal 200 is moved upward, downward, leftward and rightward under execution of the game, the X-axis magnetic sensor 312 and/or the Y-axis magnetic sensor 314 embedded in the electronic compass module 214 generate sensor output signals proportional to magnitudes of the external geomagnetic field, respectively, and transmit same to the ADC 322. The ADC 322 converts the received analog sensor output signals to digital signals and transfers same to the compensation processor 330.

The compensation processor 330 receives the digital signals from the ADC 322, analyzes the received digital signal and determines if the compensation work is required. If the compensation is determined to be required, the compensation processor 330 uses the compensation algorithms embedded therein to perform the compensation work for the digital signals and to -output the compensated digital signals to the microprocessor 220 as control data.

The microprocessor 220 of the mobile communication terminal 200 uses the control data to control a movement of the user-controlled character in the stand-alone mobile game under execution (S708). The microprocessor 220 also outputs a game screen including the movement of the user-controlled character through the LCD 213 on a real-time basis (S710).

Meanwhile, if the selected mobile game is determined to be a network mobile game in accordance with the determination result of the step S702, the microprocessor 220 is allowed to gain access to a wireless Internet game server via a wireless Internet (S712). The wireless Internet game server receives an execution request for the game from the mobile communication terminal 200 connected to the wireless Internet game server and executes the selected network mobile game (S714). Here, after the wireless Internet game server executes the network mobile game, it maintains a standby state for receiving the control data to be transmitted from the mobile communication terminal 200 in order to control the movement of the user-controlled character.

The mobile communication terminal 200 generates the control data depending on the movement thereof and transmits the control data to the wireless Internet game server via the wireless Internet (S716). The wireless Internet game server which receives the control data via the wireless Internet controls the movement of the user-controlled character depending the received control data (S718).

The wireless Internet game server which controls the movement of the user-controlled character generates a game server, which is transmitted to the mobile communication terminal 200 via the wireless Internet on a real-time basis S702). The mobile communication terminal 200 receives the game screen via the wireless Internet and displays the game screen to the LCD 213 (S722).

While the invention has been shown and described with reference to the preferred embodiment, it will be understood by those skilled in the art that various changes and modifications may be made without departing from the spirit and scope of the invention as defined in the following claims.

### INDUSTRIAL APPLICABILITY

As described above, the conventional method of playing a mobile game by using small key buttons provided on the mobile communication terminal have many difficulties. However, in accordance with the present invention, since the user-controlled character in the mobile game is moved according to the movement of the mobile communication terminal itself, upward, downward, leftward and rightward movement of the user-controlled character can be easily controlled.

Moreover, since the push count by which the key buttons provided on a small area of a key matrix are pressed is comparatively reduced to play the mobile game in accordance with the present invention, it becomes possible to play the mobile game faster and more easily with an improved precision.

1. A mobile communication terminal for supporting a mobile game by using an electronic compass module, the mobile game being a game electronically performed by or at a mobile communication terminal, wherein the mobile game stored in the mobile communication terminal or in a wireless Internet game server is executed to play the game on a real-time basis, and an output generated from the electronic compass module embedded in the mobile communication terminal is used as direction control data for changing a movement of a user-controlled character in the mobile game under execution upward, downward, leftward and rightward from a fixed point.

2. The mobile communication terminal of claim 1, wherein the electronic compass module includes:
an X-axis magnetic sensor and a Y-axis magnetic sensor for generating an X-axis magnetic senor output signal and a Y-axis magnetic sensor output signal depending on variations in an X-axis and a Y-axis component of the external geomagnetic field, respectively;
an analog/digital converter (ADC) for receiving the X-axis and/or the Y-axis magnetic sensor signal and converting received signal into a digital signal; and
a compensation processor for receiving the digital signal from the ADC, determining whether or not a compensation of the digital signal is required, performing the compensation of the digital signal if the compensation is determined to be required, and transferring compensated digital signal to the microprocessor.

3. The mobile communication terminal of claim 2, wherein the electronic compass module further includes a constant DC voltage circuit for supplying a constant DC voltage to the X-axis magnetic sensor and the Y-axis magnetic sensor.

4. The mobile communication terminal of claim 2, wherein the compensation processor determines that the compensation is required when the received digital signal has a negative value or a value of more than or equal to 360°.

5. The mobile communication terminal of claim 2, wherein the compensation processor determines that the compensation is required when the received digital signal has a value greater than a predetermined threshold value.

6. The mobile communication terminal of claim 4 or 5, wherein the compensation processor incorporates therein a compensation algorithm for use in performing the compensation.

7. The mobile communication terminal of claim 1, wherein the microprocessor controls the user-controlled character to be moved by as much as a horizontal and/or a vertical rotation angle value.

8. The mobile communication terminal of claim 1, wherein the microprocessor controls the user-controlled character to be moved as much as a value obtained by multiplying a horizontal and/or a vertical rotation angle value by a weight.

9. The mobile communication terminal of claim 1, wherein the user-controlled character is rotated leftward and/or rightward about a point on a vertical axis of the user-controlled character in response to the movement of the mobile communication terminal.

10. The mobile communication terminal of claim 1, wherein the user-controlled character is rotated upward and/or downward about a point on a horizontal axis of the user-controlled character in response to the movement of the mobile communication terminal.

11. The mobile communication terminal of claim 1, wherein the electronic compass module is embedded in a body portion of the mobile communication terminal in an equilibrium state with the body of the mobile communication terminal.

12. The mobile communication terminal of claim 1, wherein the mobile communication terminal uses three dimensional data corresponding to a horizontal and/or a vertical directional value synchronized with a movement of the mobile communication terminal and outputted from the electronic compass module as an input date value for controlling a movement of the user controllable character.

13. The mobile communication terminal of claim 1, wherein the mobile game is a stand-alone mobile game stored in the mobile communication terminal and executed exclusively in the mobile communication terminal.

14. The mobile communication terminal of claim 1, wherein the mobile game has been stored in the mobile communication terminal and executed upon connection to a wireless Internet; or has been stored in wireless Internet game server and executed upon selection thereof and connection of the mobile communication terminal to the wireless Internet.

15. The mobile communication terminal of claim 14, wherein the mobile communication terminal gains access to the wireless Internet game server so that three dimensional data and/or key values inputted by a user under execution of the mobile game are transmitted to the wireless Internet game server via the wireless Internet.

16. The mobile communication terminal of claim 1, wherein the mobile communication terminal has stored therein wireless Internet communication parameters and protocols for communicating data with the wireless Internet game server via a wireless Internet.

17. The mobile communication terminal of claim 1, wherein the mobile communication terminal is selected a group including a PDA (personal digital assistant), a cellular phone, a hand-held PC, a GSM (global system for mobile) phone, a W-CDMA (wideband CDMA) phone, a CDMA-2000 phone and an MBS (mobile broadband system) phone.

18. A method for playing a mobile game on a mobile communication terminal supporting the mobile game by using an electronic compass module embedded therein, comprising the steps of:
(a) providing a mobile game list embedded in the mobile communication terminal and determining if a selected mobile game is a stand-alone mobile game or a network mobile game;
(b) based on the result determined in step (a), allowing the mobile communication terminal to exclusively execute the selected mobile game in the mobile communication terminal or allowing the mobile communication terminal to gain access to a wireless Internet game server via a wireless Internet so that the wireless Internet game server executes the selected mobile game;
(c) controlling a movement of a user-controlled character in the mobile game under execution based on control data which is generated depending on a movement of the mobile communication terminal; and
(d) transmitting and displaying a game screen, on which the user-controlled character is moved, to the mobile communication terminal on a real-time basis to execute the mobile game.

19. The method of claim 18, wherein, in step (a), the mobile game list is provided while being classified by the stand-alone mobile game and the network mobile game.

20. The method of claim 18, wherein step (b) further includes the step of presenting a game mode supported by the selected mobile game on the mobile communication terminal before the selected mobile game is executed.

21. The method of claim 20, wherein the game mode is one of a key matrix mode in which one or more key buttons provided on a key matrix are used to control the movement of the user-controlled character; an electronic compass mode in which the electronic compass module embedded in the mobile communication terminal is used to control the movement of the user-controlled character; and a dual mode in which the key matrix mode and the electronic compass mode are supported.

22. The method of claim 21, wherein, if the game mode is the dual mode, a mode selection screen capable of selecting one of the key matrix mode and the electronic compass mode is displayed on the mobile communication terminal.

23. The method of claim 18, wherein, in step (c), the mobile communication terminal or the wireless Internet game server is controlled to move the user-controlled character.

24. The method of claim 18, wherein, in step (c), the control data contains a horizontal rotation angle value and/or a vertical rotation angle value generated depending on the movement of the mobile communication terminal and is data which is used to control an upward, a downward, a leftward or a rightward movement of the user-controlled character on the mobile game under execution.

25. The method of claim 18, wherein, in step (d), the game screen is generated and displayed by controlling a microprocessor embedded in the mobile communication terminal or the game screen is generated from the wireless Internet game server and transmitted through a wireless Internet.
